(19) United States
(12) Patent Application Publication
(10) Pub. No.: US 2007/0082076 A1
(43) Pub. Date: Apr. 12, 2007

(54) Glutathione and coffee suppository

(76) Inventor: Spencer Feldman, Makawao, HI (US)

Correspondence Address: SPENCER FELDMAN, 2599 A Olinda Rd, Makawao, HI, 96768, US

(21) Appl. No.: 11/580,474

(22) Filed: Oct. 12, 2006

Related U.S. Application Data
(60) Provisional application No. 60/726,293, filed on Oct. 12, 2005.

(51) Int. Cl. A61K 38/05; A61K 36/185

(52) U.S. Cl. 424/776; 514/18

(57) ABSTRACT

A composition containing coffee extract and glutathione in the form of a suppository.

### PARENT CASE TEXT

This application claims priority from provisional patent application No. 60/726,293 filed Oct. 12, 2005.

### FIELD OF THE INVENTION

The present invention relates to a composition containing coffee and glutathione to be administered in suppository form.

### BACKGROUND OF THE INVENTION

This invention relates to a mixture of coffee and glutathione in a suppository form.

Glutathione S-Transferases (GSTs) are a class of enzymes that are part of the phase II biotransformation of xenobiotics. GSTs conjugate xenobiotics with glutathione for removal of xenobiotics from the body.

The detoxification of many xenobiotics is dependant on the amount of both GST and glutathione in the body. If one or both are not available in sufficient quantities, then the removal of xenobiotics will be compromised.

Coffee enemas are known to increase the Glutathione-S-Transferase family of enzymes. Supplying coffee in a suppository is more convenient than using an enema. Unlike enemas where the recipient must remain on their backs for 20 minutes while the fluid is absorbed, with a suppository, there is no fluid to be retained and normal activity can continue immediately after the insertion of the suppository.

In addition, unlike enemas, suppositories do not result in the loss of electrolytes. Finally, the optimal location for the absorption of coffee into the liver (the intended target where much of the xenobiotics detoxification is done) is the hemorrhoidal complex. With a coffee suppository, the coffee is placed directly against the hemorrhoidal complex for maximum deliver to the liver, whereas with a coffee enema, a more dilute coffee in aqueous base is spread across the entire lower colon. With an enema, much of the coffee goes into the general circulation whereas with a suppository, most of the coffee goes directly to the liver.

Oral coffee does not have the same effects as rectally delivered coffee. Orally delivered coffee does not increase GST to the same degree as rectally delivered coffee, and for those sensitive to the stimulatory effects of coffee, rectally delivered coffee does not have the same stimulatory effect that oral coffee has. Furthermore, due to the direct delivery to the intended organ (the liver) less active ingredient is required and possible side effects associated with delivery of coffee to unintended tissues is minimized.

Caffeine suppositories have been used in the past for migraine headaches as caffeine will decrease cerebral blood flow but rectal delivery of purified caffeine is not the optimal method for stimulating GST. There are many compounds found in coffee (other than caffeine), which contribute to the rise in GST found in coffee enemas.

Research groups have identified 150 aliphatic compounds including 56 carbonyl compounds and 9 sulfur containing compounds; 20 alicyclic compounds, including 10 ketones; 60 aromatic benzenoid compounds, including 16 phenols; 300 heterocyclic compounds, including 74 furans, 10 hydrofurans, 37 pyrroles, 9 pyridines, 2 quinolines, 70 pyrazines, 10 quinoxalines, 3 indoles, 23 thiophens, 3 thiophenones, 28 thiazoles, and 28 oxazoles. Clarke, R. J. The Flavour of Coffee. In Dev. Food Science. 3 B. 1986. 1-47.

It is these compounds in addition to the caffeine that helps to trigger the detoxification system and concurrent increase in GST. Thus a coffee suppository, for the purposes of detoxification, is superior to a suppository made solely from purified caffeine.

Glutathione is a tripeptide and so does not survive digestion well. Its supplementation is limited to I.V. and suppository form.

A suppository containing both coffee and glutathione allows for the optimal support of the GST/glutathione detoxification pathway by providing both ingredients to the liver (the main site of detoxification) at the same time in a convenient manner requiring no enemas or intravenous administrations.

### SUMMARY OF THE INVENTION

The present invention provides an alternative to coffee enemas and glutathione I.V.'s and glutathione suppositories. A method of administering coffee and glutathione includes forming a suppository containing coffee and glutathione.

### DETAILED DESCRIPTION OF THE INVENTION

According to this invention, a suppository is formed of a spray dried coffee extract, glutathione and a matrix. The suppository is molded in a common shape from a waxy material in which the active ingredients have been dissolved or suspended. The base material may comprise cocoa butter, glycerin, glyceryl, monopalmitate, glyceryl monostearate, hydrogenated coconut oil fatty acids and hydrogenated palm kernel oil fatty acids or polyethylene glycol. The choice of base material is a matter of ordinary skill.

Preferred Method

250 mg of spray dried coffee extract is mixed with 250 mg of glutathione and put in a cocoa butter base.

I claim:

1. A method of administering a coffee and glutathione complex to a patient, comprising the steps of: (a) forming a suppository consisting essentially of containing coffee and glutathione and (b) administering said suppository to the patient.
